(19) United States
(12) Patent Application Publication
(10) Pub. No.: US 2026/0367027 A1
(43) Pub. Date: Nov. 19, 2026

(54) GAS ANALYZING SYSTEM AND GAS ANALYZING METHOD

(71) Applicant: PANASONIC HOUSING SOLUTIONS CO., LTD, Osaka (JP)

(72) Inventors: Toshihiro SAKAMOTO, Osaka (JP); Yosuke HANAI, Osaka (JP)

(21) Appl. No.: 19/153,881

(22) PCT Filed: Feb. 2, 2024
(86) PCT No.: PCT/JP2024/003473

§ 371 Date: Aug. 5, 2025

(30) Foreign Application Priority Data
Feb. 27, 2023 (JP) 2023-028243

(51) Int. Cl. G01N 27/12; G01N 33/00

(52) U.S. Cl. CPC G01N 27/126; G01N 33/0027

(57) ABSTRACT

A gas analyzing system includes a gas sensor that includes a sensing part of which an electrical characteristic value changes in accordance with adsorption of a molecule, a temperature adjuster, a temperature controller that controls a temperature of the sensing part by controlling an operation of the temperature adjuster during a measuring period in which the gas sensor exposed to a sample gas outputs a detection signal, and an analyzer that analyzes the sample gas based on the detection signal output from the gas sensor during the measuring period. During the measuring period, the temperature controller controls the temperature of the sensing part according to a temperature setting pattern that includes a first period in which the temperature of the sensing part is set to change from a first temperature to a second temperature different from the first temperature over a fixed period of time.

### TECHNICAL FIELD

The present disclosure relates to a gas analyzing system and a gas analyzing method.

### BACKGROUND ART

A gas sensor that outputs a signal corresponding to the adsorption of molecules thereon is used, for example, to detect and analyze a gas. In order to control the adsorption of molecules onto such a gas sensor, the temperature of the gas sensor is varied to change the manner in which the molecules are adsorbed on the gas sensor.

For example, Patent Literature (PTL) 1 discloses a gas detection method that includes performing energized heating control that alternates between a first energized state in which a gas sensor is purge-heated and a second energized state in which a gas is made to be adsorbed onto the gas sensor.

### CITATION LIST

### Patent Literature [PTL 1]

Japanese Unexamined Patent Application Publication No. 2001-296265

### SUMMARY OF INVENTION

### Technical Problem

In a case in which a signal output from a gas sensor in accordance with the adsorption and desorption of molecules onto and from the gas sensor is used to analyze a gas, from the viewpoint of, for example, increasing the accuracy of the analysis, it is desirable to increase the amount of information that can be obtained from a signal output from the gas sensor.

Accordingly, the present disclosure provides a gas analyzing system and so on that make it possible to obtain an increased amount of information from a signal output from a gas sensor.

### Solution to Problem

A gas analyzing system according to one aspect of the present disclosure includes: a gas sensor that includes a sensing part of which an electrical characteristic value changes in accordance with adsorption of a molecule and outputs a signal corresponding to the electrical characteristic value; a temperature adjuster that performs at least one of heating or cooling of the sensing part; a temperature controller that controls a temperature of the sensing part by controlling an operation of the temperature adjuster during a measuring period in which the gas sensor exposed to a sample gas outputs a detection signal; and an analyzer that analyzes the sample gas based on the detection signal output from the gas sensor during the measuring period, and outputs an analysis result, wherein during the measuring period, the temperature controller controls the temperature of the sensing part according to a temperature setting pattern that includes a first period in which the temperature of the sensing part is set to change from a first temperature to a second temperature different from the first temperature over a fixed period of time.

A gas analyzing method according to one aspect of the present disclosure is a gas analyzing method of analyzing a sample gas by use of a gas sensor and a temperature adjuster, the gas sensor including a sensing part of which an electrical characteristic value changes in accordance with adsorption of a molecule and outputting a signal corresponding to the electrical characteristic value, the temperature adjuster performing at least one of heating or cooling of the sensing part, and the gas analyzing method includes: controlling a temperature of the sensing part by controlling an operation of the temperature adjuster during a measuring period in which the gas sensor exposed to a sample gas outputs a signal; and analyzing the sample gas based on the signal output from the gas sensor during the measuring period, and outputting an analysis result, wherein in the controlling of the temperature of the sensing part, during the measuring period, the temperature of the sensing part is controlled according to a temperature setting pattern that includes a first period in which the temperature of the sensing part is set to change from a first temperature to a second temperature different from the first temperature over a fixed period of time.

### Advantageous Effects of Invention

The present disclosure makes it possible to obtain an increased amount of information from a signal output from a gas sensor.

### BRIEF DESCRIPTION OF DRAWINGS

FIG. 1 is a block diagram showing a schematic configuration of a gas analyzing system according to an embodiment.

FIG. 2 is a top view showing one example of a gas sensor according to an embodiment.

FIG. 3 is a schematic diagram showing one example of a configuration of an exposurer according to an embodiment.

FIG. 4 is a flowchart showing an example of an operation of a gas analyzing system according to an embodiment.

FIG. 5 is a diagram showing one example of a temperature setting pattern.

FIG. 6 is a diagram for describing a signal to be output from a gas sensor when the temperature of a sensing part is changed in accordance with a temperature setting pattern according to a comparative example.

FIG. 7 is a diagram for describing a signal to be output from a gas sensor when the temperature of a sensing part is changed in accordance with a temperature setting pattern according to an embodiment.

FIG. 8 is a diagram showing one example of a temperature setting pattern that includes a period set such that the temperature of the sensing part is maintained constant.

FIG. 9 is a diagram showing another example of a temperature setting pattern that includes a period set such that the temperature of the sensing part is maintained constant.

FIG. 10 is a diagram showing yet another example of a temperature setting pattern that includes a period set such that the temperature of the sensing part is maintained constant.

FIG. 11 is a diagram showing one example of a temperature setting pattern that includes a period set such that the temperature of the sensing part changes stepwise.

FIG. 12 is a diagram showing another example of a temperature setting pattern that includes a period set such that the temperature of the sensing part changes stepwise.

FIG. 13 is a diagram showing one example of a temperature setting pattern that includes a period set such that the temperature of the sensing part falls.

FIG. 14 is a diagram showing one example of a temperature setting pattern that includes a period set such that the temperature of the sensing part rises and a period set such that the temperature of the sensing part falls.

### DESCRIPTION OF EMBODIMENTS

### Underlying Knowledge Forming Basis of One Aspect of the Present Disclosure

A gas sensor that outputs a signal corresponding to gas adsorption includes a sensing part whose electrical characteristic value changes in accordance with the molecule adsorption, and outputs a signal corresponding to this electrical characteristic value. In a case in which such a gas sensor is used to analyze a gas, a gas is analyzed, for example, by obtaining a signal output from the gas sensor exposed to a sample gas and by using a feature value extracted from the obtained signal. In this case, when the temperature of the sensing part is changed, the manner in which molecules become adsorbed onto the sensing part also changes. Therefore, the signal to be output from the gas sensor changes as well. As a result, this makes it possible to obtain, from the gas sensor, a signal corresponding to the type and the concentration of molecules contained in the sample gas, and this in turn makes it possible to analyze the sample gas.

However, when the temperature of the gas sensor is changed in a binary manner to obtain an output from the gas sensor as in the gas detection method disclosed in PTL 1, molecules become adsorbed onto or desorbed from the sensing part rapidly. Therefore, even when gasses are of different types, the signal output from the gas sensor changes in similar patterns. Accordingly, this makes it hard to obtain information unique to the molecules that have been adsorbed onto and desorbed from the sensing part based on the signal output from the gas sensor, and this in turn reduces the amount of information that can be obtained from the signal output from the gas sensor.

The present disclosure has been made in view of such shortcomings and provides, for example, a gas analyzing system that makes it possible to obtain an increased amount of information from a signal output from a gas sensor.

### Overview of the Present Disclosure

Hereinafter, as the overview of the present disclosure, some examples of a gas analyzing system and a gas analyzing method according to the present disclosure will be described.

A gas analyzing system according to a first aspect of the present disclosure includes a gas sensor that includes a sensing part of which an electrical characteristic value changes in accordance with adsorption of a molecule and outputs a signal corresponding to the electrical characteristic value; a temperature adjuster that performs at least one of heating or cooling of the sensing part; a temperature controller that controls a temperature of the sensing part by controlling an operation of the temperature adjuster during a measuring period in which the gas sensor exposed to a sample gas outputs a detection signal; and an analyzer that analyzes the sample gas based on the detection signal output from the gas sensor during the measuring period, and outputs an analysis result, wherein during the measuring period, the temperature controller controls the temperature of the sensing part according to a temperature setting pattern that includes a first period in which the temperature of the sensing part is set to change from a first temperature to a second temperature different from the first temperature over a fixed period of time.

According to this configuration, during the measuring period, the temperature of the sensing part changes, and the output value of the detection signal also changes due to the adsorption and desorption of molecules included in the sample gas. Here, how easily a molecule becomes adsorbed onto the sensing part is unique to the kind of the molecule, and thus the relationship between the temperature and the adsorption and desorption of a molecule onto and from the sensing part is also unique to the kind of the molecule. Therefore, molecules included in the sample gas become adsorbed onto or desorbed from the sensing part in accordance with the temperature unique to each molecule, and as the temperature of the sensing part is changed from the first temperature to the second temperature over a fixed period of time as in the configuration described above, the timing at which a molecule becomes adsorbed onto or desorbed from the sensing part also is unique to the kind of the molecule. Therefore, not only the magnitude of the change in the detection signal associated with the adsorption and desorption of molecules onto and from the sensing part, but also the timing at which the detection signal changes can be used as information for analyzing the sample gas. Accordingly, the configuration described above makes it possible to obtain an increased amount of information from a detection signal output from the gas sensor.

Furthermore, for example, a gas analyzing system according to a second aspect is the gas analyzing system according to the first aspect in which the first period includes a period in which the temperature of the sensing part is sent to change at a constant rate.

In this manner, since during the period in which the temperature of the sensing part changes at a constant rate, the temperature of the sensing part changes in a similar manner regardless of the temperature range, even if the kind of the molecule included in the sample gas is unknown, information corresponding to the adsorption and desorption onto or from the sensing part unique to each molecule can be obtained effectively.

Furthermore, for example, a gas analyzing system according to a third aspect is the gas analyzing system according to the second aspect in which the first period includes the period in which the temperature of the sensing part is set to change at a constant rate and a period in which the temperature of the sensing part is maintained constant.

According to this configuration, during the period in which the temperature of the sensing part is maintained constant, even with the molecules with a small difference in the temperature at which the adsorption and desorption onto or from the sensing part occurs, the difference arises easily in the timing of the adsorption and desorption of the molecules onto or from the sensing part. Accordingly, the configuration described above makes it possible to obtain an even more increased amount of information from a detection signal output from the gas sensor.

Furthermore, for example, a gas analyzing system according to a fourth aspect is the gas analyzing system according to the third aspect in which the first period includes the period in which the temperature of the sensing part is set to change at a constant rate before and after the period in which the temperature of the sensing part is maintained constant.

According to this configuration, the temperature of the sensing part is maintained constant for a period within the first period, and the rate of change in the adsorption and desorption of the molecules onto or from the sensing part slows temporarily. Therefore, this configuration makes it possible to produce a difference in the timing of the adsorption and desorption of the molecules onto or from the sensing part depending on the kinds of the molecules.

Furthermore, for example, a gas analyzing system according to a fifth aspect of the present disclosure is the gas analyzing system according to any one of the second aspect to the fourth aspect in which the constant rate is lower than or equal to 20 °C per second.

According to this configuration, the change in the temperature of the sensing part slows during the first period, and this configuration produces a larger difference in the timing of the adsorption and desorption of the molecules onto or from the sensing part depending on the kinds of the molecules. Accordingly, the configuration described above makes it possible to obtain an even more increased amount of information from a detection signal output from the gas sensor.

Furthermore, for example, a gas analyzing system according to a sixth aspect of the present disclosure is the gas analyzing system according to any one of the first aspect to the fifth aspect in which the first period includes a period in which the temperature of the sensing part is set to change stepwise at predetermined step intervals.

According to this configuration, since the temperature of the sensing part is maintained constant in each step during the first period, even with the molecules with a small difference in the temperature at which the adsorption and desorption onto or from the sensing part occurs, this configuration more easily produces a difference in the timing of the adsorption and desorption of the molecules onto or from the sensing part at each step. Accordingly, the configuration described above makes it possible to obtain an even more increased amount of information from a detection signal output from the gas sensor.

Furthermore, for example, a gas analyzing system according to a seventh aspect is the gas analyzing system according to the sixth aspect in which the predetermined step intervals are regular intervals.

According to this configuration, since each step in which the temperature of the sensing part is maintained constant is of equal length, even in a case in which the kind of the molecule included in the sample gas is unknown, information corresponding to the adsorption and desorption onto or from the sensing part unique to the molecule can be obtained effectively.

Furthermore, for example, a gas analyzing system according to an eighth aspect is the gas analyzing system according to the sixth aspect in which the predetermined step intervals are irregular intervals.

According to this configuration, it becomes possible to adjust, or for example increase, the length of the step in which the temperature of the sensing part is maintained at a temperature at which molecules become more easily adsorbed onto or desorbed from the sensing part, in accordance with the molecules to be analyzed.

Furthermore, for example, a gas analyzing system according to a ninth aspect of the present disclosure is the gas analyzing system according to any one of the first aspect to the eighth aspect in which the sensing part includes an organic composition and conductive particles that are dispersed in the organic composition.

According to this configuration, the molecules that are adsorbed onto the sensing part can be diffused in the organic composition, and thus the diffusion of the molecules also affects the adsorption and desorption of the molecule onto and from the sensing part. Since the diffusion state of the molecules changes in accordance with the change in the temperature of the sensing part, information pertaining to a change in the diffusion state of the molecules caused by the change in the temperature of the sensing part can also be obtained from a detection signal.

Furthermore, for example, a gas analyzing system according to a tenth aspect of the present disclosure is the gas analyzing system according to any one of the first aspect to the ninth aspect in which the temperature setting pattern includes, after the first period, a second period in which the temperature of the sensing part is set to change from the second temperature in a direction in which the temperature of the sensing part approaches the first temperature over a fixed period of time.

According to this configuration, information pertaining to both the adsorption of the molecules onto the sensing part and the desorption of the molecules from the sensing part can be obtained from a detection signal.

Furthermore, for example, a gas analyzing system according to an eleventh aspect of the present disclosure is the gas analyzing system according to any one of the first aspect to the tenth aspect that further includes a temperature sensor that measures the temperature of the sensing part, wherein the temperature controller performs feedback control of the temperature of the sensing part by use of the temperature of the sensing part measured by the temperature sensor.

According to this configuration, the accuracy of the control of the temperature of the sensing part can be increased. Furthermore, as the temperature controller controls the temperature of the sensing part with the use of the temperature of the sensing part measured by the temperature sensor, it becomes possible to mitigate the manufacturing variation of the temperature adjuster and/or the variation in the rate of change in the temperature of the sensing part caused by the ambient temperature of the gas sensor.

Meanwhile, a gas analyzing method according to a twelfth aspect of the present disclosure is a gas analyzing method of analyzing a sample gas by use of a gas sensor and a temperature adjuster, the gas sensor including a sensing part of which an electrical characteristic value changes in accordance with adsorption of a molecule and outputting a signal corresponding to the electrical characteristic value, the temperature adjuster performing at least one of heating or cooling of the sensing part, and the gas analyzing method includes controlling a temperature of the sensing part by controlling an operation of the temperature adjuster during a measuring period in which the gas sensor exposed to a sample gas outputs a signal; and analyzing the sample gas based on the signal output from the gas sensor during the measuring period, and outputting an analysis result, wherein in the controlling of the temperature of the sensing part, during the measuring period, the temperature of the sensing part is controlled according to a temperature setting pattern that includes a first period in which the temperature of the sensing part is set to change from a first temperature to a second temperature different from the first temperature over a fixed period of time.

As with the gas analyzing system described above, the gas analyzing method according to the present aspect also makes it possible to obtain an increased amount of information from a detection signal output from the gas sensor.

Hereinafter, some embodiments will be described in detail with reference to the drawings as appropriate. It is to be noted that the embodiments described below merely illustrate general or specific examples. The numerical values, the shapes, the materials, the constituent elements, the arrangement positions and the connection modes of the constituent elements, the steps, the order of the steps, and so on illustrated according to the following embodiments are examples and are not intended to limit the present disclosure. Among the constituent elements described according to the following embodiments, any constituents element that are not cited in the independent claims are to be construed as optional constituent elements.

Furthermore, in the present specification, terms, such as parallel, used to describe the relationship between elements, terms used to describe the shapes of elements, and any numerical ranges indicated are not to be construed only in their strict senses but are to be construed to include substantially equivalent ranges with, for example, differences of a few percentages.

Furthermore, the drawings do not necessarily provide the exact depictions. In the drawings, substantially identical configurations are given identical reference characters, and duplicate descriptions thereof will be omitted or simplified.

### Embodiment

### Configuration

First, a configuration of a gas analyzing system according to an embodiment will be described.

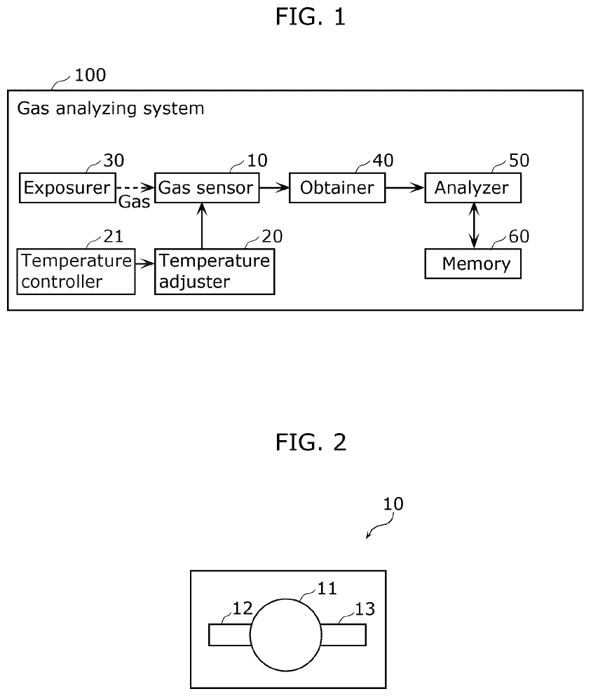

FIG. 1 is a block diagram showing a schematic configuration of gas analyzing system 100 according to the present embodiment.

As shown in FIG. 1, gas analyzing system 100 according to the present embodiment includes gas sensor 10, temperature adjuster 20, temperature controller 21, exposurer 30, obtainer 40, analyzer 50, and memory 60. Gas analyzing system 100 analyzes a sample gas based on an output of gas sensor 10 exposed to the sample gas. A sample gas includes, for example, molecules to be analyzed, such as molecules of a volatilized organic compound. A sample gas may include a plurality of kinds of molecules to be analyzed. Molecules to be analyzed may be inorganic gas molecules, such as molecules of ammonia, hydrogen sulfide, or carbon monoxide. A sample gas is, for example but is not limited to, a gas collected from a food item, an exhaled air from a human body, an air surrounding a human body, or an air sampled from a room in a building.

Gas sensor 10 is a gas sensor that outputs a signal corresponding to the adsorption of molecules onto gas sensor 10. Gas sensor 10 is, for example but is not limited to, an electrochemical gas sensor, a semiconductor gas sensor, a field effect transistor gas sensor, a surface acoustic wave gas sensor, a quartz vibrator gas sensor, or a resistive gas sensor.

Now, a specific example of gas sensor 10 will be described. FIG. 2 is a top view showing one example of gas sensor 10 according to the present embodiment. As shown in FIG. 2, gas sensor 10 includes, for example, sensing part 11 and a pair of electrodes 12 and 13 electrically connected to sensing part 11. Sensing part 11 is, for example, a sensing film whose electrical characteristic value, such as an electrical resistance value, changes in accordance with the adsorption concentration of molecules. Gas sensor 10 outputs a signal corresponding to the electrical characteristic value of sensing part 11. Specifically, gas sensor 10 outputs a signal that changes in accordance with the concentration of molecules that have been adsorbed onto sensing part 11. The signal output from gas sensor 10 may vary as different kinds of molecules become adsorbed onto sensing part 11, even when there is no difference in their adsorption concentration.

The signal corresponding to the electrical characteristic value of sensing part 11 is obtained by obtainer 40 via the pair of electrodes 12 and 13 in the form of a voltage signal or an electric current signal. For example, the electric resistance of sensing part 11 located between the pair of electrodes 12 and 13 is converted to a voltage signal or an electric current signal by a detector (not shown).

Sensing part 11 includes, for example, an organic composition that functions as an adsorber that adsorbers molecules, and conductive particles that are dispersed in the organic composition. With this configuration, as the organic composition adsorbs molecules, its volume changes, and as the manner in which the conductive particles make contact with each other changes, the electric resistance of sensing part 11 changes. The molecules that are adsorbed onto the organic composition functioning as an adsorber can be diffused into the organic composition, and thus the diffusion of the molecules also affects the adsorption and desorption of molecules onto and from sensing part 11.

The organic composition is, for example, a resin material. Non-limiting examples of such a resin material may include a polyalkylene glycol resin, a polyester resin, and a silicone resin. Such a resin material is, for example, a material that is available commercially as a stationary phase of a gas chromatography column. From the standpoint of durability and the adsorptivity of molecules, the resin material may be, for example, a silicone resin having various substituents, such as a phenyl group or a methyl group, on a side chain. Herein, the configuration of sensing part 11 is not limited to the one with an organic composition and conductive particles, and sensing part 11 may include any material whose electrical characteristic value changes in accordance with the molecule adsorption. Sensing part 11 may, for example, be constituted by an inorganic material, such as a metal oxide, or may be constituted by porous ceramics.

Gas analyzing system 100 may include a plurality of gas sensors 10. In this case, at least two gas sensors 10 of the plurality of gas sensors 10 have sensitivity characteristics different from each other. Sensing parts 11 of such at least two gas sensors 10 of the plurality of gas sensors 10 are constituted, for example, by the kinds of materials of that are different from each other. For example, such sensing parts 11 have organic compositions of different kinds. In a case in which the organic composition is a resin material, that the materials are of different kinds means, for example, that the materials substantially differ from each other in terms of at least one of the molecular weight or the composition formula. Materials of different kinds exhibit different adsorption behaviors with the same kind of molecules. In other words, at least two of the plurality of gas sensors 10 exhibit molecule adsorption behaviors different from each other. In particular, when the composition formulae of the materials differ from each other, the difference in the molecule adsorption behaviors exhibited is large. Herein, sensing parts 11 of all of the plurality of gas sensors 10 may include materials of different kinds. In this case, the plurality of gas sensors 10 output signals different from each other even when they all have adsorbed the same kind of molecules. With this configuration, the amount of information that can be obtained by gas analyzing system 100 can be further increased.

Referring back to FIG. 1, temperature adjuster 20 performs at least one of heating or cooling of sensing part 11 of gas sensor 10. Temperature adjuster 20 is, for example, a thermoelectric element that performs at least one of heating or cooling of sensing part 11. Temperature adjuster 20 may instead be a heater element that performs only the heating, or may be an element that is capable of both the heating and the cooling, such as a Peltier element. Furthermore, temperature adjuster 20 may be constituted by a combination of a plurality of elements or devices. For example, temperature adjuster 20 may include a heater and a cooling fan and thus may heat or cool sensing part 11. Furthermore, temperature adjuster 20 may be a device that heats sensing part 11 via a medium, such as a liquid.

Temperature controller 21 controls the temperature of sensing part 11. Specifically during a measuring period in which gas sensor 10 exposed to a sample gas outputs a signal, temperature controller 21 controls the operation of temperature adjuster 20 and thus controls the temperature of sensing part 11.

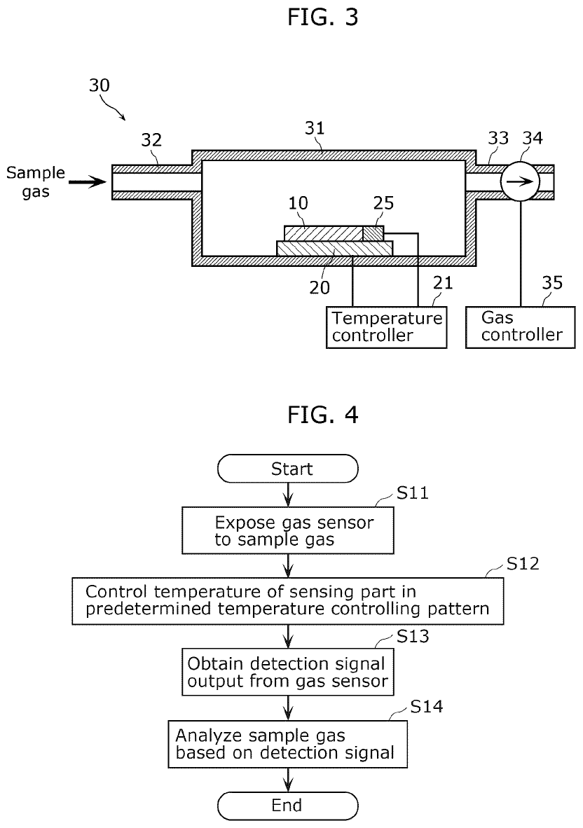

Exposurer 30 is an exposing mechanism that exposes gas sensor 10 to a gas. FIG. 3 is a schematic diagram showing one example of a configuration of exposurer 30 according to the present embodiment. As shown in FIG. 3, exposurer 30 includes, for example, housing 31, a plurality of pipes 32 and 33, intake pump 34, and gas controller 35.

Housing 31 is a box-shaped vessel that houses gas sensor 10 and temperature adjuster 20. Inside housing 31, gas sensor 10 and temperature adjuster 20 are disposed such that temperature adjuster 20 can adjust the temperature of sensing part 11 of gas sensor 10. Pipe 32 and pipe 33 are connected at their one ends to the respective ends of housing 31. As intake pump 34 operates, a gas flows from the one end of pipe 32 to the one end of pipe 33. Gas sensor 10 is disposed in the flow path through which the gas flows. Herein, in a case in which gas analyzing system 100 includes a plurality of gas sensors 10, the plurality of gas sensors 10 may be disposed, for example, in an array. In this case, furthermore, temperature adjuster 20 may be provided for each of the plurality of gas sensors 10, or single temperature adjuster 20 may be provided for the plurality of gas sensors 10.

Pipe 32 is a pipe for taking in a gas, such as a sample gas, to be analyzed from the outside of exposurer 30. For example, as intake pump 34 operates, a sample gas is taken in through pipe 32, and the sample gas that has been taken in is introduced into housing 31. Pipe 32 is provided, for example, with a solenoid valve or another type of valve for shutting off the gas flowing from the outside of exposurer 30 into housing 31.

Pipe 33 is a pipe for discharging the gas inside housing 31. For example, as intake pump 34 operates, the sample gas introduced into housing 31 is discharged to the outside of exposurer 30.

Intake pump 34 is a pump for introducing a gas into housing 31 via pipe 32 and for discharging the introduced gas via pipe 33. Intake pump 34 is provided in pipe 33. Intake pump 34 has its operation controlled by gas controller 35. Herein, intake pump 34 may instead be provided in pipe 32.

Gas controller 35 controls the operation of exposurer 30, or more specifically, controls the operation of intake pump 34. Furthermore, in a case in which a solenoid valve is provided in pipe 32, gas controller 35 may control the operation of the solenoid valve in tandem with intake pump 34. Moreover, gas controller 35 may output, to obtainer 40, information indicating the operation timing of exposurer 30, and examples of such information include information pertaining, for example, to the time or the duration of a measuring period.

It is to be noted that the configuration of exposurer 30 is not limited to the configuration shown in FIG. 3, and there is no particular limitation on the configuration as long as exposurer 30 is capable of exposing gas sensor 10 to a sample gas. For example, exposurer 30 may have a configuration in which exposurer 30 does not include intake pump 34 and in which exposurer 30 makes a carrier gas flow continuously into housing 31 and mixes the carrier gas into a sample gas. Exposurer 30 may further include various types of removal filters that remove, for example, the moisture or the fine particles from a sample gas or the like, a solenoid control valve that adjusts the flow rate in each of the pipes, or a check valve that prevents backflow in each of the pipes.

Furthermore, exposurer 30 may include a mechanism that makes it possible to introduce into housing 31 a gas other than a sample gas alternately with a sample gas. For example, pipe 32 may be branched via a three-way valve, and the gas to be introduced into housing 31 may be switched by the operation of the three-way valve. Non-limiting examples of a gas other than a sample gas may include an air for industrial use or for analysis, an inert gas, such as a nitrogen gas or a noble gas, that is substantially free of organic compounds, and a gas resulting from removing, from a sample gas, molecules by a filter that are easily adsorbed onto gas sensor 10.

As shown in FIG. 3, gas analyzing system 100 further includes temperature sensor 25 that measures the temperature of sensing part 11 of gas sensor 10. Temperature sensor 25 is disposed, for example, inside housing 31. Temperature sensor 25 is disposed in the vicinity of sensing part 11. As long as temperature sensor 25 is capable of measuring the temperature of sensing part 11, temperature sensor 25 may be in contact with sensing part 11 or may not be in contact with sensing part 11. Temperature sensor 25 is, for example is but not limited to, a thermistor, a thermocouple, or a resistance temperature detector.

Referring back to FIG. 1, obtainer 40 obtains a signal output from gas sensor 10, or more specifically, obtains a detection signal output from gas sensor 10 during a measuring period. A detection signal is a signal for analyzing a sample gas. Obtainer 40 obtains, for example, a voltage signal or an electric current signal as the signal output in accordance with the electrical characteristic value, such as an electrical resistance value, of sensing part 11 of gas sensor 10.

Analyzer 50 analyzes a sample gas based on a detection signal that obtainer 40 has obtained. When analyzing the sample gas, analyzer 50, for example, determines the air quality of the sample gas, identifies molecules included in the sample gas, or determines the smell of the sample gas.

Analyzer 50 extracts, for example, one or more feature values from the detection signal and analyzes the sample gas with the use of the extracted one or more feature values. Non-limiting examples of such a feature value include the magnitude of the detection signal at a given point of time, the amount of change in the detection signal within a given period of time, the rate of change in the detection signal within a given period of time, and the direction of change in the detection signal within a given period of time. Furthermore, the feature values to be used to analyze the sample gas may include, in addition to the one or more feature values extracted from the detection signal, the setting value or the measured value of the temperature of sensing part 11 temporally corresponding to the one or more feature values extracted from the detection signal. Moreover, the feature values to be used to analyze the sample gas may include, for example but not limited to, the outside temperature or the humidity. When analyzing the sample gas, analyzer 50 uses, for example, a trained logical data model. The trained logical data model, for example, accepts the one or more feature values extracted from the detection signal as an input, and outputs the result of analyzing the sample gas.

Analyzer 50 outputs, for example, information for displaying the result of the analysis on a display (not shown) or the like provided in gas analyzing system 100. Analyzer 50 may output such information indicating the result of the analysis to memory 60 and store this information in memory 60. Furthermore, analyzer 50 may output the information indicating the result of the analysis to an external device.

Temperature controller 21, gas controller 35, obtainer 40, and analyzer 50 are implemented with, for example but not limited to, a microcomputer or a processor having embedded therein a program for performing the processes described above and the processes described below. In this case, temperature controller 21, gas controller 35, obtainer 40, and analyzer 50 may be implemented with, for example but not limited to, separate microcomputers or processors, and two or more of these functions may be implemented with, for example but not limited to, a single microcomputer or a single processor. Furthermore, temperature controller 21, gas controller 35, obtainer 40, and analyzer 50 may each be implemented with a logic circuit dedicated to performing the processes described above and the processes described below.

Memory 60 is a storage device that stores a trained logical data model used by analyzer 50. Memory 60 is implemented, for example, with a semiconductor memory.

The trained logical data model is a logical data model for analyzing a sample gas. The trained logical data model, for example, accepts the one or more feature values extracted from the detection signal as an input, and outputs the result of analyzing the sample gas.

The trained logical data model is constructed, for example, through machine learning in which the training data includes the existing results of analyses and the one or more feature values extracted from the detection signal obtained when the sample gases producing the existing results of the analyses are used. For the logical data model in machine learning, a neural network, a random forest, a support vector machine, or a self-organizing map, for example, is used.

### Operation

Next, an operation (a process) of gas analyzing system 100 according to the present embodiment, or in other words, a gas analyzing method will be described.

FIG. 4 is a flowchart showing an example of an operation of gas analyzing system 100 according to the present embodiment.

As shown in FIG. 4, exposurer 30 exposes gas sensor 10 to a sample gas (step S11). For example, exposurer 30 exposes gas sensor 10 to a sample gas by operating intake pump 34 in accordance with the control of gas controller 35.

Next during a measuring period in which gas sensor 10 exposed to the sample gas outputs a signal, temperature controller 21 controls the operation of temperature adjuster 20 and thus controls the temperature of sensing part 11 according to a predetermined temperature setting pattern (step S12). In the measuring period, gas sensor 10 may continue to be exposed to the sample gas or may be exposed to a gas other than the sample gas. Furthermore, gas sensor 10 may start being exposed to the sample gas, coinciding with the start of the measuring period. In other words, step S11 and step S12 may start simultaneously. Furthermore, in a case in which gas sensor 10 continues to be exposed to the sample gas during the measuring period, the sample gas may be made to continue to circulate within housing 31, for example, by keeping intake pump 34 in operation, or the sample gas may be made to remain still within housing 31 by stopping the operation of intake pump 34. The measuring period is, for example, longer than or equal to 3 seconds and shorter than or equal to 60 seconds.

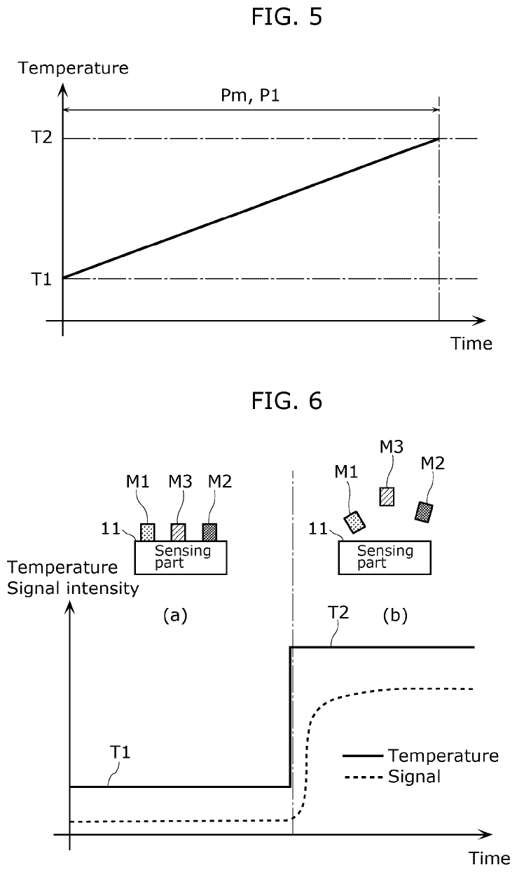

FIG. 5 is a diagram showing one example of the temperature setting pattern. In FIG. 5, the vertical axis represents the setting value for controlling the temperature of sensing part 11, and the horizontal axis represents the time. Temperature controller 21 controls the operation of temperature adjuster 20, for example, such that the temperature of sensing part 11 changes according to the temperature setting pattern shown in FIG. 5. It is to be noted that the actual temperature of sensing part 11 can deviate from the temperature setting pattern shown in FIG. 5 due to the precision of the temperature adjustment.

As shown in FIG. 5, the temperature setting pattern according to which the temperature of sensing part 11 is controlled in measuring period Pm includes first period P1 in which the temperature of sensing part 11 changes from first temperature T1 to second temperature T2 different from first temperature T1 over a fixed period of time. In the example shown in FIG. 5, the start and the end of first period P1 coincide with the start and the end of measuring period Pm in which a detection signal is output from gas sensor 10. First period P1 is, for example, longer than or equal to 1 second and shorter than or equal to 60 seconds.

First temperature T1 is the temperature of sensing part 11 held at the start of first period P1, and second temperature T2 is the temperature of sensing part 11 held at the end of first period P1. First temperature T1 may be the temperature of sensing part 11 held immediately before the start of measuring period Pm and may be a temperature higher than or lower than the temperature of sensing part 11 held immediately before the start of measuring period Pm. Meanwhile, second temperature T2 is a temperature at which the state of adsorption of molecules included in the sample gas onto sensing part 11 is substantially different from the state observed at first temperature T1. First temperature T1 and second temperature T2 are set as appropriate in accordance with, for example, the composition of a sample gas to be analyzed as well as the heat resistance, the cold resistance, and the like of gas sensor 10. The difference between first temperature T1 and second temperature T2 is smaller than the maximum range of temperature change that can be produced by temperature adjuster 20 within first period P1. The difference between first temperature T1 and second temperature T2 is, for example, greater than or equal to 10 °C and smaller than or equal to 150 °C.

In the example shown in FIG. 5, second temperature T2 is higher than first temperature T1, and first period P1 is a period set such that the temperature of sensing part 11 rises. Furthermore, in first period P1, the temperature of sensing part 11 changes constantly in the same direction, showing a monotonous rise without ever falling. Herein, first period P1 may instead be a period in which the temperature of sensing part 11 is controlled to fall. A specific example of such first period P1 will be described later.

Furthermore, in the example shown in FIG. 5, first period P1 is a period set such that the temperature of sensing part 11 changes at a constant rate. In other words, the slope of the change in the temperature of sensing part 11 with respect to time is linear, and thus the temperature of sensing part 11 changes linearly. The constant rate in this case (the rate of rise in the temperature in the example shown in FIG. 5) is, for example, lower than or equal to 20 °C per second, from the standpoint of increasing the amount of information to be obtained from a detection signal. Furthermore, the constant rate in this case is, for example, higher than or equal to 1 °C per second. Moreover, in first period P1, this constant rate is smaller than the maximum rate of change in the temperature of sensing part 11 that can be achieved by temperature adjuster 20 when the temperature is changed in the same direction. In the present specification, the rate at which the temperature changes is expressed by an absolute value, which is not affected by the direction of change in the temperature. Herein, the rate of change in the temperature of sensing part 11 may be varied within first period P1. As long as the temperature continues to change within first period P1, for example, the slope of the change in the temperature of sensing part 11 with respect to the time may vary within first period P1, or the temperature of sensing part 11 may change curvilinearly.

Temperature controller 21, for example, performs feedback control of the temperature of sensing part 11 so as to achieve the temperature setting pattern described above with the use of the temperature of sensing part 11 measured by temperature sensor 25. This feedback control can increase the accuracy of control of the temperature of sensing part 11. Furthermore, as temperature controller 21 controls the temperature of sensing part 11 with the use of the temperature of sensing part 11 measured by temperature sensor 25, it becomes possible to mitigate the manufacturing variation of temperature adjuster 20 and/or the variation in the rate of change in the temperature of sensing part 11 caused by the ambient temperature of gas sensor 10.

There is no particular limitation on the method of the feedback control. The feedback control may be ON/OFF control, and may be control that includes at least proportional control. The control that includes at least proportional control may be P control in which only proportional control is performed, PI control in which proportional control and integral control are performed, PD control in which proportional control and derivative control are performed, or PID control in which proportional control, integral control, and derivative control are performed.

Referring back to FIG. 4, next, obtainer 40 obtains a detection signal output from gas sensor 10 in measuring period Pm (step S13). Then, analyzer 50 analyzes the sample gas based on the detection signal obtained by obtainer 40 (step S14). Analyzer 50, for example, extracts one or more feature values from the obtained detection signal and analyzes the sample gas based on the extracted one or more feature values. Analyzer 50, with the use of the trained logical data model, accepts the extracted one or more feature values as an input and outputs the result of analyzing the sample gas. The result of analyzing the sample gas shows, for example but not limited to, the determination of the air quality of the sample gas, the determination as to whether the molecules to be analyzed are included in the sample gas, the identification result of molecules included in the sample gas, or the determination result of the smell of the sample gas.

### Advantageous Effects

Now, some advantageous effects of gas analyzing system 100 according to the present embodiment will be described.

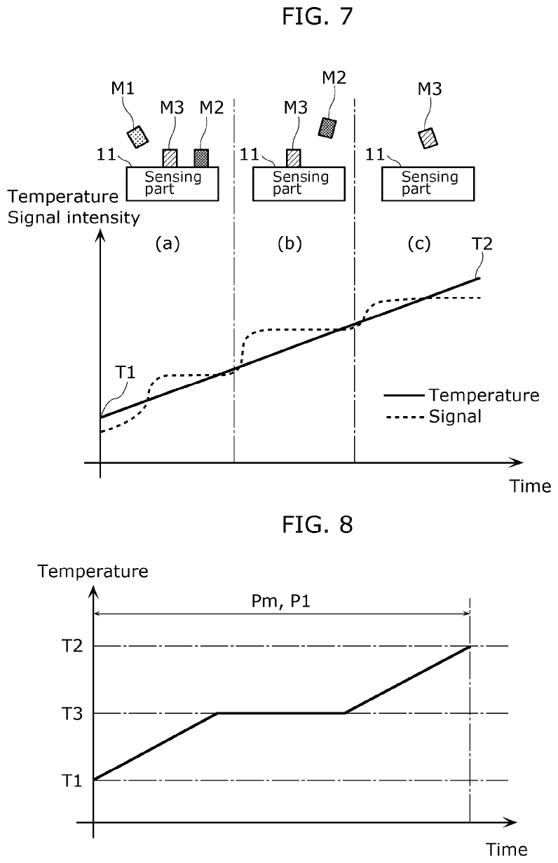

FIG. 6 is a diagram for describing a signal to be output from gas sensor 10 when the temperature of sensing part 11 is changed in accordance with a temperature setting pattern according to a comparative example. FIG. 7 is a diagram for describing a signal to be output from gas sensor 10 when the temperature of sensing part 11 is changed in accordance with a temperature setting pattern according to the present embodiment. FIG. 6 shows the change over time in the intensity of the signal output from gas sensor 10 observed when the temperature of sensing part 11 is controlled so as to change in a binary manner from first temperature T1 to second temperature T2. Meanwhile, FIG. 7 shows the change over time in the intensity of the signal output from gas sensor 10 observed when the temperature of sensing part 11 is controlled according to the temperature setting pattern shown in FIG. 5. In the graphs shown in FIG. 6 and FIG. 7, the vertical axis represents the setting value of the temperature of sensing part 11 or the intensity of the signal output from gas sensor 10, and the horizontal axis represents the time.

Furthermore, the portions above the graphs shown in FIG. 6 and FIG. 7 schematically show the change in the manner in which molecules are adsorbed onto sensing part 11 in the measuring period. FIG. 6 and FIG. 7 show a case in which a sample gas includes three kinds of molecules, namely, molecules M1 to M3. Molecules M1 to M3 differ from one another in how easily they interact with sensing part 11 (e.g., the magnitude of the intermolecular force between the molecule and the sensing part). Among molecules M1 to M3, molecule M1 interacts with sensing part 11 least easily, molecule M2 interacts with sensing part 11 the next least easily, and molecule M3 interact with sensing part 11 most easily. In other words, among molecules M1 to M3, molecule M1 becomes adsorbed onto sensing part 11 least easily, and molecule M3 becomes adsorbed onto sensing part 11 most easily.

As the temperature of sensing part 11 rises, the kinetic energy of the molecules adsorbed on sensing part 11 increases. Therefore, the molecules adsorbed on sensing part 11 become desorbed more easily from sensing part 11. For example, when the force of the kinetic energy of a molecule adsorbed on sensing part 11 exceeds the intermolecular force between this molecule and sensing part 11, this molecule becomes desorbed from sensing part 11.

As shown in FIG. 6, in the temperature setting pattern according to the comparative example, molecules M1 to M3 are adsorbed on sensing part 11 during the period marked by (a) in FIG. 6, in which the temperature of sensing part 11 is constant at first temperature T1. Thereafter, in the period marked by (b) in FIG. 6, which immediately follows the point at which the temperature of sensing part 11 has changed to second temperature T2, the kinetic energy of all three molecules M1 to M3 exceeds their respective intermolecular forces with sensing part 11, and molecules M1 to M3 all become desorbed from sensing part 11. Accordingly, the intensity of the detection signal output from gas sensor 10 changes rapidly, and thus individualized information of molecules M1 to M3 become unavailable from the detection signal, and the only information that can be obtained from the detection signal is information concerning the desorption of all three molecules M1 to M3. It is to be noted that, in FIG. 6 and FIG. 7, the intensity of the signal output from gas sensor 10 increases as the molecules become desorbed, but the intensity of the signal output from gas sensor 10 may instead decrease as the molecules become desorbed.

In contrast, as shown in FIG. 7, in the temperature setting pattern according to the present embodiment, the temperature of sensing part 11 rises from first temperature T1 to second temperature T2 over a fixed period of time. Therefore, in the period marked by (a) in FIG. 7, in which the temperature of sensing part 11 is relatively low, first, only molecule M1, which becomes adsorbed onto sensing part 11 least easily, becomes desorbed from sensing part 11. Next, in the period marked by (b) in FIG. 7, in which the temperature has risen, molecule M2, which becomes adsorbed onto sensing part 11 the next least easily, becomes desorbed from sensing part 11. Lastly, in the period marked by (c) in FIG. 7, in which the temperature has further risen, molecule M3, which becomes adsorbed onto sensing part 11 most easily, also becomes desorbed from sensing part 11. In this manner, with the temperature setting pattern according to the present embodiment, molecules M1 to M3 become desorbed from sensing part 11 sequentially, and the intensity of the detection signal output from gas sensor 10 changes stepwise. This stepwise change in the intensity of the detection signal corresponds to molecules M1 to M3, and this configuration, therefore, makes it possible to obtain individualized information of molecules M1 to M3. Accordingly, gas analyzing system 100 according to the present embodiment makes it possible to obtain an increased amount of information from a detection signal. As a result, for example, gas analyzing system 100 according to the present embodiment makes it possible to increase the accuracy of analysis or the number of items that can be analyzed, when a sample gas is analyzed by analyzer 50. Furthermore, even in a case in which a sample gas includes only one or two of molecules M1 to M3, gas analyzing system 100 according to the present embodiment makes it possible to obtain the magnitude of the change in the intensity of a detection signal and the timing of the change of the intensity of the detection signal as information unique to the corresponding molecule or molecules.

Although an example in which the temperature of sensing part 11 rises over a fixed period of time has been described with reference to FIG. 7, when the temperature of sensing part 11 falls over a fixed period of time, the phenomenon opposite to what has been described above occurs. In other words, molecules M3 to M1 sequentially become adsorbed onto sensing part 11, and the intensity of the detection signal output from gas sensor 10 changes stepwise. Therefore, even in a case in which the temperature of sensing part 11 falls over a fixed period of time, it is possible to obtain an increased amount of information from the detection signal.

### Other Examples of Temperature Setting Pattern

At step S12 described above, the temperature setting pattern used by temperature controller 21 to control the temperature of sensing part 11 is not limited to the example shown in FIG. 5. There is no particular limitation on the temperature setting pattern as long as the temperature setting pattern includes first period P1 set such that the temperature of sensing part 11 changes from first temperature T1 to second temperature T2 different from first temperature T1 over a fixed period of time. In the following, other examples of the temperature setting pattern will be described. It is to be noted that the following description centers on the differences from the example of the temperature setting pattern shown in FIG. 5, and the description of features shared therebetween will be omitted or simplified.

First, some examples of a temperature setting pattern that includes a period set such that the temperature of sensing part 11 is maintained constant will be described.

FIG. 8 to FIG. 10 are each a diagram showing one example of a temperature setting pattern that includes a period set such that the temperature of sensing part 11 is maintained constant. In each of FIG. 8 to FIG. 10, the vertical axis represents the setting value for controlling the temperature of sensing part 11, and the horizontal axis represents the time.

As shown in FIG. 8 to FIG. 10, first period P1 may include a period set such that the temperature of sensing part 11 changes at a constant rate and a period set such that the temperature of sensing part 11 is maintained constant. The constant rate is as described with reference to FIG. 5. Furthermore, in the examples shown in FIG. 8 to FIG. 10, second temperature T2 is higher than first temperature T1, and first period P1 is a period in which the temperature of sensing part 11 is controlled so as to rise. In addition, in the examples shown in FIG. 8 to FIG. 10, the temperature of sensing part 11 during first period P1 rises monotonously without ever falling. Herein, as long as the temperature of sensing part 11 continues to change in the period set such that the temperature of sensing part 11 changes at a constant rate, instead of the temperature of sensing part 11 changing at a constant rate, for example, the slope of the change in the temperature of sensing part 11 with respect to time may change, or the temperature of sensing part 11 may change curvilinearly. In other words, in the examples shown in FIG. 8 to FIG. 10, first period P1 may include a period set such that the temperature of sensing part 11 changes unidirectionally and a period in which the temperature of sensing part 11 is maintained constant.

In the example shown in FIG. 8, first period P1 includes, before and after the period set such that the temperature of sensing part 11 is maintained constant at third temperature T3, a period set such that the temperature of sensing part 11 changes at a constant rate. Third temperature T3 is a temperature between first temperature T1 and second temperature T2 and is different from first temperature T1 and second temperature T2. According to the temperature setting pattern shown in FIG. 8, the temperature of sensing part 11 first changes from first temperature T1 to third temperature T3 at a constant rate, is then maintained at third temperature T3, and then changes from third temperature T3 to second temperature T2 at a constant rate. Third temperature T3 is set as appropriate in accordance with, for example but not limited to, the composition of a sample gas to be analyzed.

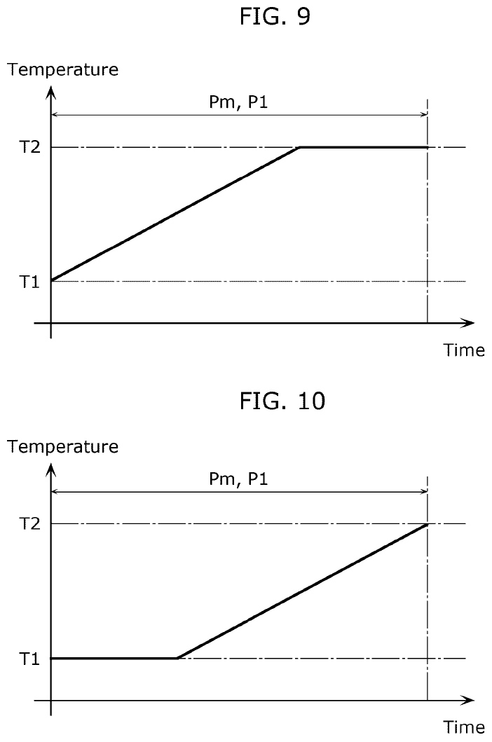

Meanwhile, in the examples shown in FIG. 9 and FIG. 10, first period P1 includes, only either before and after the period set such that the temperature of sensing part 11 is maintained constant at third temperature T3, a period set such that the temperature of sensing part 11 changes at a constant rate.

In the temperature setting pattern shown in FIG. 9, the temperature of sensing part 11 is first maintained at first temperature T1 and then changes from first temperature T1 to second temperature T2 at a constant rate. In the example shown in FIG. 9, first temperature T1 is a temperature higher than or lower than the temperature of sensing part 11 held immediately before measuring period Pm. Alternatively, first temperature T1 may be the same as the temperature of sensing part 11 held immediately before measuring period Pm.

Meanwhile, according to the temperature setting pattern shown in FIG. 10, the temperature of sensing part 11 first changes from first temperature T1 to sensing part 11 at a constant rate and is then maintained at second temperature T2.

Now, some advantageous effects of first period P1 including a period set such that the temperature is maintained constant will be described. The adsorption and desorption of molecules onto and from sensing part 11 is an equilibrium reaction in which the equilibrium constant changes due to a change in the temperature of sensing part 11. Furthermore, a change in the manner in which molecules are adsorbed onto and desorbed from sensing part 11 requires time for the equilibrium to shift. For example, in the case of raising the temperature of sensing part 11, it is not that the molecules of the same kind become desorbed at once when the temperature reaches a certain level but even the molecules of the same kind become desorbed over some period of time. Therefore, when the temperature of sensing part 11 continues to rise, the desorption is accelerated, and thus molecules with a small difference in how easily they become adsorbed onto sensing part 11 become desorbed at similar timings. As shown in FIG. 8 to FIG. 10, when first period P1 includes a period set such that the temperature of sensing part 11 is maintained constant, since there is no acceleration in the desorption rate in this period, even the molecules with a small difference in how easily they become adsorbed onto sensing part 11 become less likely to have different desorption timings. As a result, even in a case in which a sample gas includes molecules with a small difference in how easily they become adsorbed onto sensing part 11, it becomes easier to obtain individualized information of each of these molecules from the detection signal output from gas sensor 10.

Furthermore, when the temperature of sensing part 11 is maintained constant, the equilibrium constant remains constant, and thus the behavior for shifting to the equilibrium state of the adsorption and desorption of the molecules at this temperature is observed in the detection signal output from gas sensor 10. In other words, the behavior of the adsorption and desorption of the molecules onto or from sensing part 11 in the equilibrium state unique to each of these molecules allows the fluctuation of the detection signal unique of each of the molecules to be observed. Accordingly, the configuration described above makes it possible to obtain an even more increased amount of information to be obtained from a detection signal.

Furthermore, in the example shown in FIG. 8, the temperature of sensing part 11 is maintained constant for a duration while the temperature of sensing part 11 is raised in first period P1. Therefore, in first period P1, the rate of change in the desorption of molecules from sensing part 11 temporarily slows during that period. Accordingly, this configuration makes it possible to effectively vary the timings of desorption, from sensing part 11, of molecules with a small difference in how easily they become adsorbed onto sensing part 11.

Although the examples shown in FIG. 8 to FIG. 10 include one period, within first period P1, set such that the temperature of sensing part 11 is maintained constant, there may be two or more such periods within first period P1.

Next, some examples of a temperature setting pattern that includes a period set such that the temperature of sensing part 11 changes stepwise will be described.

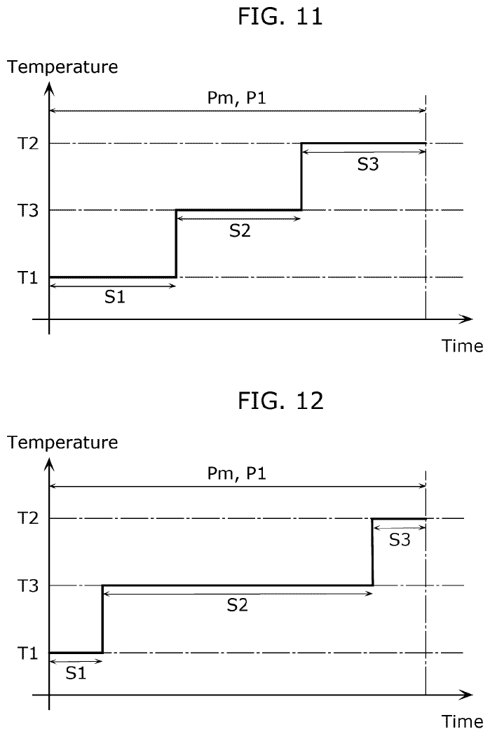

FIG. 11 and FIG. 12 are each a diagram showing one example of a temperature setting pattern that includes a period set such that the temperature of sensing part 11 changes stepwise. In each of FIG. 11 and FIG. 12, the vertical axis represents the setting value for controlling the temperature of sensing part 11, and the horizontal axis represents the time.

As shown in FIG. 11 and FIG. 12, first period P1 may include a period set such that the temperature of sensing part 11 changes stepwise at predetermined step intervals. Furthermore, in the examples shown in FIG. 11 and FIG. 12, second temperature T2 is higher than first temperature T1, and first period P1 is a period in which the temperature of sensing part 11 is controlled so as to rise. In addition, in the examples shown in FIG. 11 and FIG. 12, the temperature of sensing part 11 during first period P1 rises monotonously without ever falling. Herein, although entire first period P1 is the period set such that the temperature of sensing part 11 changes stepwise in the examples shown in FIG. 11 and FIG. 12, these are not limiting examples. For example, first period P1 may include a period set such that the temperature of sensing part 11 changes stepwise and a period set such that the temperature of sensing part 11 changes at a constant rate.

In the examples shown in FIG. 11 and FIG. 12, the temperature of sensing part 11 changes stepwise between three levels in the order of from first temperature T1, to third temperature T3, and to second temperature T2. When the temperature of sensing part 11 is in which the temperature of sensing part 11 is set to change stepwise in this manner, in first period P1, the temperature of sensing part 11 changes from first temperature T1 to second temperature T2 with third temperature T3 intervening between first temperature T1 and second temperature T2. First temperature T1 is, for example, a temperature higher than or lower than the temperature of sensing part 11 held immediately before measuring period Pm. Alternatively, first temperature T1 may be the same as the temperature of sensing part 11 held immediately before measuring period Pm. Third temperature T3 is set as appropriate in accordance with, for example but not limited to, the composition of a sample gas to be analyzed. Herein, the number of levels of the stepwise change is not limited to three, and the temperature may change between four or more levels.

In the example shown in FIG. 11, the predetermined step intervals of the stepwise change are regular intervals. In other words, period S1 in which the temperature of sensing part 11 is maintained at first temperature T1, period S2 in which the temperature of sensing part 11 is maintained at third temperature T3, and period S3 in which the temperature of sensing part 11 is maintained at second temperature T2 are equal to each other in duration.

Meanwhile, in the example shown in FIG. 12, the predetermined step intervals of the stepwise change are irregular intervals. In other words, at least one of period S1 in which the temperature of sensing part 11 is maintained at first temperature T1, period S2 in which the temperature of sensing part 11 is maintained at third temperature T3, and period S3 in which the temperature of sensing part 11 is maintained at second temperature T2 differs from the other period or periods in duration. Herein, in the example shown in FIG. 12, among periods S1 to S3, period S2 in which the temperature of sensing part 11 is maintained at third temperature T3 is the longest.

As first period P1 includes a period set such that the temperature of sensing part 11 changes stepwise at predetermined step intervals in this manner, the temperature of sensing part 11 is maintained constant in each step. Therefore, as with the examples shown in FIG. 8 to FIG. 10, even the molecules with a small difference in how easily they become adsorbed onto sensing part 11 become less likely to have different desorption timings. As a result, even in a case in which a sample gas includes molecules with a small difference in how easily they become adsorbed onto sensing part 11, it becomes easier to obtain individualized information of each of these molecules from the detection signal output from gas sensor 10. Furthermore, the behavior for shifting to the equilibrium state of the adsorption and desorption of molecules at the temperature maintained constant is observed in the detection signal output from gas sensor 10. Accordingly, the configuration described above makes it possible to obtain an even more increased amount of information to be obtained from a detection signal. Furthermore, since the temperature of sensing part 11 is controlled so as to change stepwise, the process of the control by temperature controller 21 can be simplified.

Next, an example of a temperature setting pattern that includes a period set such that the temperature of sensing part 11 falls will be described.

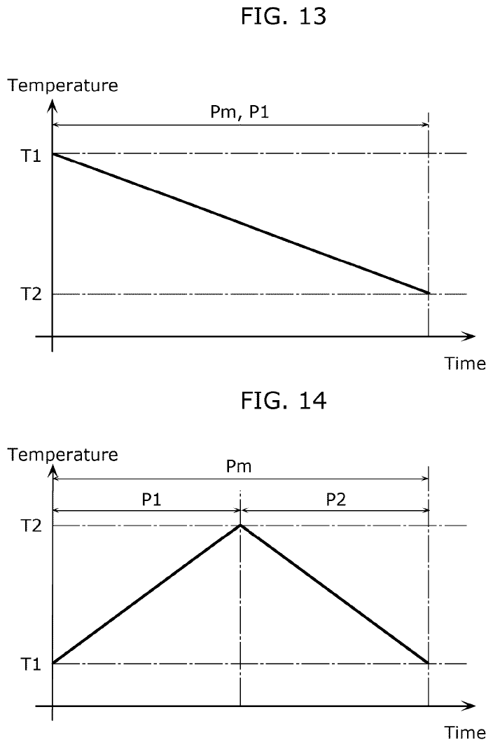

FIG. 13 is a diagram showing one example of a temperature setting pattern that includes a period set such that the temperature of sensing part 11 falls. In FIG. 13, the vertical axis represents the setting value for controlling the temperature of sensing part 11, and the horizontal axis represents the time.

As shown in FIG. 13, first period P1 may be a period set such that the temperature of sensing part 11 falls. Therefore, in the example shown in FIG. 13, second temperature T2 is lower than first temperature T1. Furthermore, in first period P1, the temperature of sensing part 11 changes constantly in the same direction, showing a monotonous fall without ever rising. Furthermore, in the example shown in FIG. 13, first period P1 is a period set such that the temperature of sensing part 11 changes at a constant rate. In other words, in the example shown in FIG. 13, the high and low relationship between first temperature T1 and second temperature T2 in the temperature setting pattern shown in FIG. 5 is reversed. Herein, the high and low relationship between first temperature T1 and second temperature T2 may be reversed also in the examples of the temperature setting patterns shown in FIG. 8 to FIG. 12.

Next, an example of a temperature setting pattern that includes a period set such that the temperature of sensing part 11 rises and a period set such that the temperature of sensing part 11 falls will be described.

FIG. 14 is a diagram showing one example of a temperature setting pattern that includes a period set such that the temperature of sensing part 11 rises and a period set such that the temperature of sensing part 11 falls. In FIG. 14, the vertical axis represents the setting value for controlling the temperature of sensing part 11, and the horizontal axis represents the time.

As shown in FIG. 14, the temperature setting pattern may include, after first period P1 set such that the temperature of sensing part 11 changes from first temperature T1 to second temperature T2 over a fixed period of time, second period P2 set such that the temperature of sensing part 11 changes from second temperature T2 to first temperature T1 in the direction in which the temperature of sensing part 11 approaches first temperature T1 over a fixed period of time. The example shown in FIG. 14 is an example in which second period P2 is further added after first period P1 of the temperature setting pattern shown in FIG. 5.

In the example shown in FIG. 14, the temperature of sensing part 11 changes from second temperature T2 to first temperature T1 in second period P2. The temperature of sensing part 11 held at the end of second period P2 is not limited to first temperature T1 and may be higher than or lower than first temperature T1 as long as that temperature is closer to first temperature T1 than to second temperature T2. Furthermore, in the example shown in FIG. 14, first period P1 starts at the same time as the start of measuring period Pm, second period P2 starts immediately after the end of first period P1, and measuring period Pm ends at the same time as the end of second period P2.

As the temperature setting pattern includes first period P1 and second period P2 in this manner, molecules become adsorbed onto sensing part 11 and also become desorbed from sensing part 11 in measuring period Pm. Accordingly, a detection signal that corresponds to the adsorption of molecules and a detection signal that corresponds to the desorption of molecules can both be obtained, and this configuration makes it possible to obtain an even more increased amount of information from a detection signal.

Herein, when a temperature setting pattern includes first period P1 and second period P2, the example in which second period P2 is added after first period P1 of the temperature setting pattern shown in FIG. 5 is not a limiting example. Such a temperature setting pattern may be a temperature setting pattern in which second period P2 corresponding to first period P1 is added after that first period P1 of the temperature setting pattern shown in any of FIG. 8 to FIG. 12.

Furthermore, even in a case in which a temperature setting pattern includes first period P1 and second period P2, second temperature T2 may be higher than first temperature T1 as shown in FIG. 5, FIG. 8 to FIG. 12, and FIG. 14, or second temperature T2 may be lower than first temperature T1 as shown in FIG. 13.

In addition, the number of temperature setting patterns to be executed by temperature controller 21 is not limited to one. For example, temperature controller 21 may select one temperature setting pattern from a plurality of temperature setting patterns that differ from each other, and may control the temperature or sensing part 11 according to the selected temperature setting pattern. For example, temperature controller 21 selects one temperature setting pattern based on an input operation performed by a user selecting a temperature setting pattern for the control input to an input interface (not shown).

### Other Embodiments

Thus far, the gas analyzing system and the gas analyzing method according to the present disclosure have been described based on some embodiments, but these embodiments do not limit the present disclosure. Unless departing from the spirit of the present disclosure, an embodiment obtained by making various modifications that are conceivable by a person skilled in the art to the embodiments or another embodiment constructed by combining some of the constituent elements of the embodiments is also encompassed by the scope of the present disclosure.

While gas analyzing system 100 according to the foregoing embodiments includes exposurer 30, gas analyzing system 100 does not have to include exposurer 30. For example, gas sensor 10 may be exposed to a sample gas as gas sensor 10 is disposed in a space in which a sample gas is present.

Furthermore, while gas analyzing system 100 according to the foregoing embodiments include temperature sensor 25, gas analyzing system 100, for example, does not have to include temperature sensor 25. In this case, for example, a condition for controlling temperature adjuster 20 such that the temperature of sensing part 11 changes according to a predetermined temperature setting pattern may be obtained in advance through an experiment or the like, and temperature controller 21 may control the operation of temperature adjuster 20 in accordance with this controlling condition.

Furthermore, while obtainer 40 according to the foregoing embodiments directly obtains a signal output from gas sensor 10, this configuration, for example, is not a limiting example. For example, obtainer 40 may include a communication circuit and may obtain a signal output from gas sensor 10 via a wide area communication network, such as the internet.

Furthermore, gas analyzing system 100 according to the foregoing embodiments may be implemented in the form of a single gas analyzing device that includes the constituent elements described above or may be implemented by a plurality of devices. In a case in which gas analyzing system 100 is implemented by a plurality of devices, the constituent elements of gas analyzing system 100 may be distributed to the plurality of devices in any ways within an implementable range.

Furthermore, for example, according to the foregoing embodiments, the whole or a part of the constituent elements of the gas analyzing system according to the present disclosure may be implemented with a dedicated piece of hardware or may be realized through the execution of software programs suitable for the respective constituent elements. Each of the constituent elements may be implemented as a program executing unit, such as a central processing unit (CPU) or a processor, reads out a software program recorded in a recording medium, such as a hard disk drive (HDD) or a semiconductor memory, and executes the software program.

The constituent elements of the gas analyzing system according to the present disclosure may be implemented with one or more electronic circuits. The one or more electronic circuits may each be a general purpose circuit or a dedicated circuit.

The one or more electronic circuits may include, for example but not limited to, a semiconductor device, an integrated circuit (IC), or a large scale integration (LSI) circuit. An IC or an LSI circuit may be integrated into a single chip or into a plurality of chips. Although the term used herein is IC or LSI circuit, a circuit may also be called a system LSI circuit, a very large scale integration (VLSI) circuit, or an ultra large scale integration (ULSI) circuit depending on the degree of integration. A field programmable gate array (FPGA) that can be programmed after manufacturing an LSI circuit can also be used for the same purpose.

A general or specific aspect of the present disclosure may be implemented in the form of a system, a device, a method, an integrated circuit, or a computer program. Alternatively, a general and specific aspect may be implemented in the form of a non-transitory computer readable recording medium, such as an optical disc, an HDD, or a semiconductor memory, storing the computer program. Furthermore, a general or specific aspect may be implemented through a desired combination of a system, a device, a method, an integrated circuit, a computer program, and a recording medium.

For example, the present disclosure may be realized in the form of a gas analyzing method to be executed by a computer, such as a gas analyzing system, or may be realized in the form of a program that causes a computer to execute such a gas analyzing method. Furthermore, the present disclosure may be realized in the form of a non-transitory computer readable recording medium having such a program recorded thereon.

### Industrial Applicability

The gas analyzing system and the gas analyzing method according to the present disclosure are useful, for example, in determining the air quality or the smell or in identifying molecules in a gas.

### Reference Signs List

10 gas sensor
20 temperature adjuster
21 temperature controller
25 temperature sensor
30 exposurer
31 housing
32, 33 pipe
34 intake pump
35 gas controller
40 obtainer
50 analyzer
60 memory
100 gas analyzing system

1. A gas analyzing system comprising:
a gas sensor that includes a sensing part of which an electrical characteristic value changes in accordance with adsorption of a molecule and outputs a signal corresponding to the electrical characteristic value;
a temperature adjuster that performs at least one of heating or cooling of the sensing part;
a temperature controller that controls a temperature of the sensing part by controlling an operation of the temperature adjuster during a measuring period in which the gas sensor exposed to a sample gas outputs a detection signal; and
an analyzer that analyzes the sample gas based on the detection signal output from the gas sensor during the measuring period, and outputs an analysis result, wherein
during the measuring period, the temperature controller controls the temperature of the sensing part according to a temperature setting pattern that includes a first period in which the temperature of the sensing part is set to change from a first temperature to a second temperature different from the first temperature over a fixed period of time.

2. The gas analyzing system according to claim 1, wherein
the first period includes a period in which the temperature of the sensing part is set to change at a constant rate.

3. The gas analyzing system according to claim 2, wherein
the first period includes the period in which the temperature of the sensing part is set to change at the constant rate and a period in which the temperature of the sensing part is maintained constant.

4. The gas analyzing system according to claim 3, wherein
the first period includes the period in which the temperature of the sensing part is set to change at the constant rate before and after the period in which the temperature of the sensing part is maintained constant.

5. The gas analyzing system according to claim 2, wherein
the constant rate is lower than or equal to 20 °C per second.

6. The gas analyzing system according to claim 1, wherein
the first period includes a period in which the temperature of the sensing part is set to change stepwise at predetermined step intervals.

7. The gas analyzing system according to claim 6, wherein
the predetermined step intervals are regular intervals.

8. The gas analyzing system according to claim 6, wherein
the predetermined step intervals are irregular intervals.

9. The gas analyzing system according to claim 1, wherein
the sensing part includes an organic composition and conductive particles dispersed in the organic composition.

10. The gas analyzing system according to claim 1, wherein
the temperature setting pattern includes, after the first period, a second period in which the temperature of the sensing part is set to change from the second temperature in a direction in which the temperature of the sensing part approaches the first temperature over a fixed period of time.

11. The gas analyzing system according to claim 1, further comprising:
a temperature sensor that measures the temperature of the sensing part, wherein
the temperature controller performs feedback control of the temperature of the sensing part by use of the temperature of the sensing part measured by the temperature sensor.

12. A gas analyzing method of analyzing a sample gas by use of a gas sensor and a temperature adjuster, the gas sensor including a sensing part of which electrical characteristic value changes in accordance with adsorption of a molecule and outputting a signal corresponding to the electrical characteristic value, the temperature adjuster performing at least one of heating or cooling of the sensing part, the gas analyzing method comprising:
controlling a temperature of the sensing part by controlling an operation of the temperature adjuster during a measuring period in which the gas sensor exposed to a sample gas outputs a signal; and
analyzing the sample gas based on the signal output from the gas sensor during the measuring period, and outputting an analysis result, wherein
in the controlling of the temperature of the sensing part, during the measuring period, the temperature of the sensing part is controlled according to a temperature setting pattern that includes a first period in which the temperature of the sensing part is set to change from a first temperature to a second temperature different from the first temperature over a fixed period of time.
